(19) United States
(12) Patent Application Publication
(10) Pub. No.: US 2003/0050491 A1
(43) Pub. Date: Mar. 13, 2003

(54) Process for removing unsaponifiable materials from a fatty acid

(76) Inventors: Michael D. Sublett, Cincinnati, OH (US); Christopher G. Ciolino, Cincinnati, OH (US)

Correspondence Address: COGNIS CORPORATION, 2500 RENAISSANCE BLVD., SUITE 200, GULPH MILLS, PA, 19406

(21) Appl. No.: 09/951,047

(22) Filed: Sep. 12, 2001

(51) Int. Cl. C07C 57/12

(52) U.S. Cl. 554/176

(57) ABSTRACT

A process for purifying a fatty acid containing unwanted unsaponifiable materials involving: (a) providing a C₄-C₂₆ fatty acid starting material containing unsaponifiable materials; (b) reacting the fatty acid starting material with a zinc oxide component to form a zinc soap; (d) heating the zinc soap; (e) distilling the unsaponifiable materials from the zinc soap; (f) cooling the purified zinc soap; (g) adding a neutralizing acid component to the purified zinc soap to form a mixture containing a zinc salt and an organic layer containing clean fatty acid; and (h) distilling the purified fatty acid from the organic layer.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

Dimer acids and their preparation have been known for some time. Processes for dimerizing monounsaturated fatty acids, such as oleic acid and polyunsaturated acids, such as linoleic and linolenic acid, a mixture of which with oleic acid is found in tall oil acids, are well known.

Dimer acids are used in a wide variety of products such as surface coatings, unsaturated polyesters, elastomers, lubricants and greases, plasticizers and epoxy resins.

The first step in the production of dimer acids is the oligomerization of the unsaturated fatty acids producing a mixture referred to as the “Total Product”. The “Total Product” is a blend of monomer acids, dibasic acids, and polybasic acids. Monomer acids are a mixture of unreacted saturated acids, isomerized unsaturated acids, and isomerized saturated acids. The monomer acids are typically separated from the dimer acids by distillation.

The monomer acids are then typically hydrogenated and solvent separated, yielding a liquid phase and a solid phase. The liquid phase containing isostearic acid, also contains the unsaponifiable materials. It is because of the presence of these unsaponifiable materials that the acid value of this isostearic acid is less than 190, rendering it much more difficult to sell on the open market.

### BRIEF SUMMARY OF THE INVENTION

The present invention is directed to a process for continuously purifying a fatty acid containing unwanted unsaponifiable materials, thereby raising its acid value, the process involving:

(a) providing a C₄-C₂₆ fatty acid starting material containing unsaponifiable materials;

(b) providing a zinc oxide component;

(c) reacting the fatty acid starting material with the zinc oxide component to form a zinc soap;

(d) heating the zinc soap;

(e) distilling the unsaponifiable materials from the zinc soap to form a purified zinc soap;

(f) cooling the purified zinc soap to a temperature of less than 100° C.;

(g) providing a low boiling neutralizing acid with a pKa less than about 4.0;

(h) adding the acid component to the purified zinc soap to form a mixture containing a zinc salt that precipitates out and an organic phase containing purified fatty acid;

(i) separating the zinc salt from the organic phase by filtration;

(j) recirculating the zinc salt into step (b) of the process.

### BRIEF DESCRIPTION OF THE SEVERAL VIEWS OF THE DRAWING

Not applicable.

### DETAILED DESCRIPTION OF THE INVENTION

Other than in the operating examples, or where otherwise indicated, all numbers expressing quantities of ingredients or reaction conditions are understood as being modified, in all instances, by the term “about”.

The present invention is directed to the surprising discovery that unsaponifiable materials can be continuously removed from low acid value fatty acids, such as isostearic acid having an acid value of less than 190, in a manner which minimizes the degradation of the fatty acid starting material during the purification process, thereby maintaining high theoretical yields of the purified product. Moreover, the process lends itself to being used in a continuous manner with minimal loss of purifying materials, due to the ability of the process to re-use/recycle said purifying materials in a continuous fashion.

The chemical components employed by the present process include: (1) a C₄-C₂₆ fatty acid starting material containing unsaponifiable materials; (2) a zinc oxide purifying component; and (3) a low-boiling neutralizing acid component having a pKa of less than about 4.0.

The starting material used in the process of the present invention can be any C₄-C₂₆ fatty acid containing unsaponifiable materials. Preferred fatty acid starting materials include methyl-branched saturated C₁₄-C₂₄ fatty acids obtained from hydrogenated industrial fatty acid mixtures.

Methyl-branched, saturated and unsaturated C₁₄-C₂₄ fatty acids occur as a by-product in the thermal or catalytic dimerization of the corresponding unsaturated straight chain fatty acids. One example thereof, which also represents a preferred fatty acid starting material for use in the process of the present invention, are the isostearic acids formed in the dimerization of tall oil fatty acids. The isostearic acids, which are present in the monomeric fatty acid fraction produced by the dimerization process, are rich in unsaponifiable materials, such as hydrocarbons and other unreactive materials, which have virtually no acid functionality. Due to the presence of these unsaponifiable materials in the isostearic acids, their acid values are low, generally in the range of from 170 to 175. In order to increase the acid value of these fatty acids to a more useable/acceptable level, the aforementioned unsaponifiable materials must be removed. Thus, fatty acid starting materials such as these are prime candidates for purification by the process of the present invention.

Another critical component of the process of the present invention is zinc oxide which reacts with the fatty acid starting material to form zinc soap. While other types of soaps such as sodium, potassium and calcium may be used in order to remove unsaponified materials from a fatty acid in order to purify it, these soaps are solid even at very high temperatures, i.e., greater than 150° C. Consequently, acidulation of these types of soaps is very difficult, leading to a reduction in yield of purified fatty acid. The advantage associated with the use of zinc, however, stems from its soap remaining in liquid form at temperatures below 100° C., thereby enabling it to be more easily and effectively neutralized in subsequent acidulating steps.

The final critical component employed in the process of the present invention is the acid component, used to acidulate the zinc soap once formed. The acid component, in general, can be any acid having a pKa of less than about 4.0. Examples thereof include, but are not limited to, chloroacetic, hydroxyacetic, and formic acids.

The process of the present invention will be described in reference to removing unwanted unsaponifiable materials from isostearic acid, wherein the isostearic acid is obtained from the monomeric fatty acid fraction obtained from the dimerization of tall oil fatty acid, it being understood, however, that any suitable fatty acid starting material containing unwanted unsaponifiable materials may be employed. Thus, according to one embodiment of the process of the present invention, the isostearic acid starting material containing unsaps is first reacted with an effective amount of zinc oxide to form zinc isostearate. The zinc isostearate is then heated, under 1 mm Hg vacuum, to a temperature of from about 180 to about 260° C., and preferably about 240° C., thereby distilling the unwanted unsaponifiable materials from the zinc isostearate. Once all of the unsaponifiable materials have been distilled off, the zinc isostearate is then cooled to a temperature of from about 60 to about 100° C., and preferably about 90° C., the zinc isostearate remaining in liquid form. An effective amount of an acid is then added to the liquid zinc isostearate in order to acidulate the soap to an acid value of from about 180 to about 220. This acidulation step results in the formation of zinc salts which can subsequently be filtered from the solution. The isostearic acid is then distilled, yielding a purified isostearic acid having an acid value greater than 190.

The zinc salt collected by, for example, filtration is then re-used by mixing it with a fresh batch of fatty acid starting material in order to remove unwanted unsaponifiable materials therefrom. It may be necessary, however, to combine some additional zinc oxide with the recirculated zinc salt in order to completely effectuate its reaction with the isostearic acid containing unsaponifiable materials into zinc isostearate. As the zinc isostearate is forming, the low boiling fatty acid may also be removed/distilled from the mixture and re-used back in the process. As the distillation temperature is increased, all of the neutralizing acid is removed and isolated. As the temperature of distillation continues upward, the unsaponifiable materials are next to distill. When the distillation of the unsaponifiable materials is complete, the temperature of the zinc salts is lowered to less than 100° C. The neutralizing acid is again added, and the zinc salt of the neutralizing acid is formed and precipitates out. The zinc salts are removed by filtration, and the organic acids remaining are purified by distillation yielding a product with an acid value greater than 190.

It is thus seen that the process of the present invention not only provides an effective means for removing unsaponifiable materials from a fatty acid, but is also efficient due to the continuous re-usability of the zinc salt by-product of the process.

According to yet another embodiment of the present invention, it has been surprisingly discovered that by employing a neutralizing acid having a pKa of less than about 4.0 one can eliminate the need for any water washing steps subsequent to the addition of the neutralizing acid to the zinc soap. Moreover, the zinc salt will then more completely precipitate out of solution and, once filtered, can immediately be re-introduced into the process for the treatment of fresh unsap-containing fatty acid starting material.

Thus, according to this aspect of the invention, in the above-described process, formic acid is used as the neutralizing acid since its pKa of 3.8 is significantly less than that of the isostearic acid thereby enabling the zinc salt to more completely precipitate out of solution. Once the acid is added, zinc formate precipitates out from the organic solution. The zinc formate is then filtered and re-introduced back to the beginning of the process for the purification of new unsap-containing isostearic acid starting material. The remaining organic phase need not be water washed thereafter. Hence, by employing a neutralizing acid having a pKa less than 4.0, substantial savings in both time and materials are realized.

The present invention will be better understood from the examples which follow, all of which are intended for illustrative purposes only, and are not meant to unduly limit the scope of the invention in any way.

### EXAMPLES

### Example 1

EMERY 935 Fatty Acid (649.0 g, 2.00 moles), a mixture of straight chain saturated acids and isostearic acids with an initial acid value of 172.8, was combined with zinc oxide (85.0 g, 1.040 moles) in a two liter 4-neck round bottom flask. Attached to the flask were a thermometer well for controlling temperature, a nitrogen inlet tube for providing an inert atmosphere, a motorized stirrer assembly, and a Claisen head attached to a condenser, a vacuum take-off, and a 500 ml round bottom receiver. This mixture was heated under a nitrogen atmosphere to 220° C. and held for 20 minutes to form the zinc soap of the fatty acid. The water from the reaction was continuously distilled off. The zinc isostearate was cooled to 138° C., at which time the system was put under vacuum (less than 5 Torr). Heat was applied to the zinc isostearate. At 180° C. (0.3 Torr), the unsaponifiable material began to distill. Heating was continued until the zinc isostearate temperature reached 250° C. This temperature was held until the distillation of the unsaponifiable material stopped. The zinc isostearate was then cooled to 90° C. under high vacuum.

The vacuum was then removed, and formic acid (99.7 g, 2.08 moles) was added to neutralize the zinc soap forming isostearic acid and the precipitate, zinc formate. The mixture was cooled to 70° C. and filtered to separate the isostearic acid from the zinc formate. The zinc formate was then saved for future reactions (see Example 2). The initial acid value of the EMERY®935 Fatty Acid was 172.8. After the treatment described above, the acid value was raised to 197.2.

### Example 2

2.00 moles (645 g) of EMERY 935 Fatty Acid (initial acid value of 174.0) were combined with 1.020 moles (191 g) of zinc formate generated in Example 1 and recycled here in Example 2. In an identical manner to Example 1, the mixture was heated to 228° C. and held for 20 minutes. The contents was then cooled, vacuum was applied and the unsaponifiable materials were removed by distillation. The contents was then again cooled, vacuum removed, and 2.040 moles of formic acid were added to precipitate the zinc formate and regenerate the EMERY®935 Fatty Acid. The zinc formate was removed by filtration and again saved for further use.

The acid value of the treated product was 194.9.

### Example 3

Same as example 2 except that EMERSOL 871 Isostearic Acid having an initial acid value of 179.0 was combined with 1.020 moles of zinc oxide. The acid value of the treated product was 199.3.

### Examples 4-7

Each example used 2.00 moles of the same batch of EMERSOL®871 Isostearic Acid and 2.040 moles of formic acid.

### Example 4

1.020 moles of the zinc formate from example 3 was recycled and added to virgin isostearic acid. After treatment, zinc formate was regenerated and the EMERSOL®871 Isostearic Acid had an acid value of 196.2.

### Example 5

1.020 moles of the zinc formate from example 4 was recycled and added to virgin isostearic acid. After treatment, zinc formate was regenerated and the EMERSOL®871 Isostearic Acid had an acid value of 196.9.

### Example 6

1.020 moles of the zinc formate from example 5 was recycled and added to virgin isostearic acid. After treatment, zinc formate was regenerated and the EMERSOL®871 Isostearic Acid had an acid value of 193.7.

### Example 7

1.020 moles of the zinc formate from example 6 was recycled and added to virgin isostearic acid. After treatment, zinc formate was regenerated and the EMERSOL®871 Isostearic Acid had an acid value of 196.4.

What is claimed is:

1. A process for purifying a fatty acid containing unwanted unsaponifiable materials, thereby raising its acid value, the process comprising:
(a) providing a C₄-C₂₆ fatty acid starting material containing unsaponifiable materials;
(b) providing a zinc oxide component;
(c) reacting the fatty acid starting material with the zinc oxide component to form a zinc soap;
(d) heating the zinc soap;
(e) distilling the unsaponifiable materials from the zinc soap to form a purified zinc soap;
(f) cooling the purified zinc soap to a temperature of less than 100° C.;
(g) providing a low-boiling neutralizing acid component having a pKa of less than about 4.0;
(h) adding the acid component to the purified zinc soap to form a mixture containing a zinc salt and an organic layer containing clean fatty acid;
(i) separating the zinc salt from the organic layer; and
(j) recirculating the zinc salt into step (b) of the process.

2. The process of claim 1 wherein the fatty acid starting material is a methyl-branched saturated C₁₄₋₂₄ fatty acid.

3. The process of claim 1 wherein the fatty acid starting material is isostearic acid.

4. The process of claim 1 wherein the fatty acid starting material has an acid value in a range of from about 170 to about 175.

5. The process of claim 1 wherein the zinc soap is heated to a temperature of from about 180 to about 260° C.

6. The process of claim 1 further comprising distilling out the low-boiling neutralizing acid having a pKa of less than about 4.0 from the organic layer and recirculating it into step (g) of the process.

7. A process for purifying a fatty acid containing unwanted unsaponifiable materials, thereby raising its acid value, the process comprising:
(a) providing a C₁₄-C₂₄ fatty acid starting material containing unsaponifiable materials;
(b) providing a zinc oxide component;
(c) reacting the fatty acid starting material with the zinc oxide component to form a zinc soap;
(d) heating the zinc soap;
(e) distilling the unsaponifiable materials from the zinc soap to form a purified zinc soap;
(f) cooling the purified zinc soap to a temperature of less than 100° C.;
(g) providing a neutralizing acid component having a pKa similar to that of the fatty acid starting material;
(h) adding the acid component to the purified zinc soap to form a mixture containing a zinc salt and an organic layer containing clean fatty acid;
(i) filtering out the zinc salt from the organic layer;
(j) distilling the purified fatty acid from the organic layer; and
(k) introducing the zinc salt into step (b) of the process.

8. The process of claim 7 wherein the fatty acid starting material is a methyl-branched saturated C₁₄₋₂₄ fatty acid.

9. The process of claim 7 wherein the fatty acid starting material is isostearic acid.

10. The process of claim 7 wherein the fatty acid starting material has an acid value in a range of from about 170 to about 175.

11. The process of claim 7 wherein the zinc soap is heated to a temperature of about 240° C.

12. The process of claim 9 wherein the neutralizing acid component is formic acid.

13. The product of the process of claim 1.

14. The product of the process of claim 2.

15. The product of the process of claim 3.

16. The product of the process of claim 4.

17. The product of the process of claim 5.

18. The product of the process of claim 7.

19. The product of the process of claim 8.

20. The product of the process of claim 9.

21. The product of the process of claim 10.

22. The product of the process of claim 11.

23. The product of the process of claim 12.
